(19) United States
(12) Patent Application Publication
(10) Pub. No.: US 2026/0294871 A1
(43) Pub. Date: Oct. 1, 2026

(54) PHARMACEUTICAL COMBINATIONS AND COMPOSITIONS THEREOF COMPRISING ANTIBACTERIAL AGENTS

(71) Applicant: Cipla Limited, Mumbai (IN)

(72) Inventors: Vaishali Vikas GUPTE, Mumbai (IN); Jaideep Ashok GOGTAY, Mumbai (IN); Sandesh Vishwanath SAWANT, Mumbai (IN)

(21) Appl. No.: 19/480,577

(22) PCT Filed: May 2, 2024
(86) PCT No.: PCT/IB2024/054239

§ 371 Date: Oct. 31, 2025

(30) Foreign Application Priority Data
May 2, 2023 (IN) 202321031203

(51) Int. Cl. A61K 31/407; A61K 31/198; A61K 31/439; A61P 31/04

(52) U.S. Cl. CPC A61K 31/407; A61K 31/198; A61K 31/439; A61P 31/04

(57) ABSTRACT

The present invention relates to the combination of meropenem and one or more antibiotic resistance breakers, pharmaceutical compositions containing the same, and methods for treating bacterial infections that include administering the same. Particularly, it relates to a pharmaceutical composition comprising (a) meropenem, (b) avibactam, and (c) EDTA.

### FIELD OF INVENTION

The invention relates to the combination of meropenem and one or more antibiotic resistance breakers, pharmaceutical compositions containing the same, and methods for treating bacterial infections that include administering the same.

### BACKGROUND OF INVENTION

Bacterial infections continue to remain one of the major causes contributing towards human diseases. One of the key challenges in the treatment of bacterial infections is the ability of bacteria to develop resistance to one or more antibacterial agents over time. Examples of such Multidrug-resistant (MDR) and carbapenem-resistant (CR) bacteria that have developed resistance to typical antibacterial agents include: Escherichia coli, Klebsiella, Acinetobacter etc. The problem of emerging drug-resistance in bacteria is often tackled by switching to newer antibacterial agents, which can be more expensive and sometimes more toxic. Additionally, this may not be a permanent solution as the bacteria often develop resistance to the newer antibacterial agents over time. In general, bacteria are particularly efficient in developing resistance, because of their ability to multiply very rapidly and pass on the resistance genes as they replicate.

Treatment of infections caused by resistant bacteria remains a key challenge for the health care professionals.

Arora et. al reports the Microbiological Efficacy of Meropenem-EDTA Combination in a Tertiary Care Intensive Care Unit. (Arora, Charu Dutt, et al. “Microbiological Efficacy Of Meropenem-Ethylenediaminetetraacetic Acid Combination As Compared To Meropenem In A Tertiary Care Intensive Care Unit.” (2019).

Hafi et. al reports evaluating the Efficacies of Carbapenem/b-Lactamase Inhibitors against Carbapenem-Resistant Gram-Negative Bacteria in vitro and in vivo. (El Hafi, Bassam, et al. “Evaluating the efficacies of carbapenem/β-lactamase inhibitors against carbapenem-resistant gram-negative bacteria in vitro and in vivo.” Frontiers in Microbiology 10 (2019): 933).

IN212237 reports heterocyclic compounds which are active as inhibitors of beta lactamases in association with an antibiotic of beta-lactamine type.

There is a long felt need for development of newer ways to treat infections that are becoming resistant to known therapies and methods. The present inventors have surprisingly developed pharmaceutical combinations and compositions thereof having antibacterial activity even against highly resistant bacterial strains.

### OBJECTS OF THE INVENTION

It is an object of the present invention to provide a combination of meropenem and one or more antibiotic resistance breakers. The combination can be present, e.g., in a bulk form. Alternatively, the combination can be present in a pharmaceutical composition. The pharmaceutical composition can further include one or more pharmaceutically acceptable excipients.

It is another object of the present invention to provide a process for preparing a pharmaceutical combination that includes meropenem and one or more antibiotic resistance breakers.

It is another object of the present invention to provide a pharmaceutical composition that includes the combination of meropenem and one or more antibiotic resistance breakers, along with one or more pharmaceutically acceptable excipients.

It is yet another object of the present invention to provide a method for treating a bacterial infection in a subject, which method includes administering to said subject an effective amount of a combination of meropenem and one or more antibiotic resistance breakers. The combination can be present in a pharmaceutical composition.

It is yet another object of the present invention to provide a combination of meropenem and one or more antibiotic resistance breakers, for use in treating a bacterial infection. The combination can be present in a pharmaceutical composition. The pharmaceutical composition can further include one or more pharmaceutically acceptable excipients.

### SUMMARY OF THE INVENTION

According to an aspect of the present invention, there is provided a combination of (a) meropenem and (b) one or more antibiotic resistance breakers. The combination can be present, e.g., in a bulk form. Alternatively, the combination can be present in a pharmaceutical composition. The pharmaceutical composition can further include one or more pharmaceutically acceptable excipients.

According to another aspect of the present invention, there is provided a combination of (a) meropenem and (b) one or more antibiotic resistance breakers. The one or more antibiotic resistance breakers can include, e.g., avibactam, and EDTA.

According to another aspect of the present invention, there is provided a pharmaceutical composition that includes the combination of (a) meropenem, (b) one or more antibiotic resistance breakers such as avibactam EDTA, and (c) one or more pharmaceutically acceptable excipients.

According to yet another aspect of the present invention, there is provided a method for treating bacterial infection in a subject. The method includes administering to said subject an effective amount of a combination that includes (a) meropenem, (b) avibactam, and EDTA. The combination can be present as a pharmaceutical composition. The pharmaceutical composition can further include one or more pharmaceutically acceptable excipients.

### DETAILED DESCRIPTION OF THE INVENTION

The following description is provided to assist in a comprehensive understanding of exemplary embodiments of the invention. It includes various specific details to assist in that understanding, but these are to be regarded as merely exemplary.

Accordingly, those skilled in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the scope of the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the terms “comprises/comprising” and “include/includes” when used is taken to specify the presence of stated features, steps or components but does not preclude the presence or addition of one or more other features, steps, components, or groups thereof.

The present invention relates to pharmaceutical combinations and compositions thereof comprising meropenem and one or more antibiotic resistance breakers and methods for treating highly resistant bacterial infections.

The term “antibiotic resistance breakers” as used herein includes non-antibiotic moieties which do not have any antimicrobial activity on its own, but, in combination with antibiotics enhance their antimicrobial activity and help overcome resistance barriers.

The term “infection” as used herein refers to the multiplication of parasitic organisms within the human body; invasion of the body by pathogenic microorganisms that reproduce and multiply, causing disease by local cellular injury, secretion of a toxin, or antigen-antibody reaction in the host; a disease caused by the invasion of the body by pathogenic microorganisms. This will exclude normal bacterial flora of the intestinal tract.

The term “bacterial infection” as used herein refers to the multiplication of bacteria within the human body; an infection as described herein, caused by bacteria.

The term “treat,” “treating,” or “treatment” as used herein refers to administration of the combination described herein, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to treating a subject already suffering from the infection. The terms “treat”, “treating,” or “treatment” as used herein also refer to administering the combination described herein, with or without additional pharmaceutically active or inert ingredients, in order to: (i) reduce or eliminate a bacterial infection, one or more symptoms of a bacterial infection, or a combination thereof, or (ii) retard progression of a bacterial infection, or one or more symptoms of a bacterial infection, or a combination thereof, or (iii) reduce the severity of a bacterial infection, or one or more symptoms of a bacterial infection, or a combination thereof, or (iv) suppress clinical manifestation of a bacterial infection, or (v) suppress manifestation of adverse symptoms of a bacterial infection.

The terms “pharmaceutically effective amount” or “therapeutically effective amount” or “effective amount” as used herein refer to an amount of the combination described herein, which has a therapeutic effect, or is the amount required to produce a therapeutic effect in a subject. For example, a “therapeutically effective amount” or “pharmaceutically effective amount” or “effective amount” of the combination described herein is the amount required to produce a desired therapeutic effect as may be judged by clinical trial results, model animal infection studies, and/or in vitro studies (e.g. in agar or broth media). Such effective amount depends on several factors, including but not limited to, the microorganism (e.g. bacteria) involved, characteristics of the subject (for example height, weight, sex, age and medical history), severity of infection and particular type of the antibacterial agent used. For prophylactic treatments, a prophylactically effective amount is that amount which would be effective in preventing the bacterial infection.

The term “administration” or “administering” refers to and includes delivery of the combination described herein to a subject, including for example, by any appropriate method, which serves to deliver the combination to the site of infection. The combination can be present in a pharmaceutical composition and the pharmaceutical composition can further include one or more pharmaceutically acceptable excipients. The method of administration may vary depending on various factors, such as for example, the type, nature, and amount of the components of the combination, including the meropenem as well as the one or more antibiotic resistance breakers and any inert ingredients (excipients), the site of the potential or actual infection, the microorganism involved, the severity of the infection, the age and physical condition of the subject, and the like. Suitable routes to administer a pharmaceutical composition containing the combination described herein include, e.g., oral, intravenous, topical, intrarespiratory, intraperitoneal, intramuscular, parenteral, sublingual, transdermal, intranasal, aerosol, intraocular, intratracheal, intrarectal, and vaginal. Suitable dosage forms of the pharmaceutical composition containing the combination described herein include, e.g., gene gun, dermal patch, eye drop, and mouthwash. When the combination described herein is formulated as a pharmaceutical composition, one of the ways of administering such pharmaceutical composition is by admixing the ingredients (e.g., in the form of a suitable unit dosage form, such as tablet, capsule, solution, powder or a like) and then administering to the subject the dosage form. Alternatively, the ingredients may also be administered separately (simultaneously or one after the other) as long as these ingredients achieve beneficial therapeutic levels, such that the pharmaceutical composition as a whole provides a synergistic and/or desired effect.

The term “growth” as used herein refers to a growth of one or more microorganisms and includes reproduction or population expansion of the microorganism (e.g. A. baumannii, E. coli, K. pneumoniae, P. aeruginosa, E. cloacae). The term “growth” also includes maintenance of on-going metabolic processes of the microorganism, including the processes that keep the microorganism alive.

The term, “effectiveness” as used herein refers to ability of a treatment, a pharmaceutical composition, or pharmaceutically active ingredients, to produce a desired biological effect in a subject. For example, the term “antibacterial effectiveness” of a composition or of an antibacterial agent refers to the ability of the composition or the antibacterial agent to prevent or treat bacterial infection in a subject.

The term “synergistic” or “synergy” as used herein refers to the interaction of two or more agents so that their combined effect is greater than their individual effects.

The term “antibacterial agent” as used herein refers to any substance, compound, combination of substances, or combination of compounds capable of: (i) inhibiting, reducing, or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of a bacteria.

The term “beta-lactam antibacterial agent” as used herein refers to compounds with antibacterial properties and containing a beta-lactam nucleus in their molecular structure. Typical, non-limiting examples include penicillins, cephalosporins, monobactams and carbapenems.

The term “beta-lactamase” or “beta-lactamase enzyme” as used herein refers to any enzyme or protein or any other substance that breaks down a beta-lactam ring. The term “beta-lactamase” includes enzymes that are produced by bacteria and have the ability to hydrolyse the beta-lactam ring in a beta-lactam compound, either partially or completely.

The term “extended spectrum beta-lactamase” (ESBL) as used herein includes those betalactamase enzymes, which are capable of conferring bacterial resistance to various beta-lactam antibacterial agents, such as penicillins, cephalosporins, aztreonam, and the like.

The term “carbapenamases” as used herein refers to Beta lactamases which can hydrolyze carbapenems.

The term “beta-lactamase inhibitor” as used herein refers to a compound capable of inhibiting activity of one or more beta-lactamase enzymes, either partially or completely. Typical, non-limiting examples include avibactum, sulbactum and clavulanic acid.

The term “colony forming units” or “CFU” as used herein refers to an estimate of number of viable bacterial cells per ml of the sample. Typically, a “colony of bacteria” refers to a mass of individual bacteria growing together.

The term “meropenem” as used herein refers to a carbapenem antibiotic used to treat a variety of bacterial infections. The spectrum of action includes many Gram-positive and Gram-negative bacteria (including Pseudomonas) and anaerobic bacteria. Meropenem has the IUPAC name (4R,5S,6S)-3-(((3S,5S)-5-(dimethylcarbamoyl) pyrrolidin-3-yl)thio)-6-((R)-1-hydroxyethyl)-4-methyl-7-oxo-1-azabicyclo[3.2.0]hept-2-ene-2-carboxylic acid; CAS number 119478-56-7; chemical formula C₁₇H₂₅N₃O₅S; and molar mass 383.46 g·mol⁻¹. Meropenem can exist in the unionized form, or as a pharmaceutically acceptable salt thereof. As such, the term “meropenem” encompasses the compound in the unionized form, as well as pharmaceutically acceptable salts thereof. The structural formula is shown below.

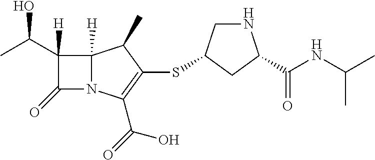 [SMILES: CC(C)NC(=O)[C@@H]1C[C@H](SC2=C(C(=O)O)N3C(=O)[C@H]([C@@H](C)O)[C@H]3[C@H]2C)CN1]

The term “avibactam” as used herein refers to a non-β-lactam β-lactamase inhibitor having the IUPAC name [(2S,5R)-2-carbamoyl-7-oxo-1,6-diazabicyclo[3.2.1]octan-6-yl] hydrogen sulfate and CAS number 1192500-31-4. Avibactam has the chemical formula C₇H₁₁N₃O₆S and molar mass 265.24 g·mol⁻¹. Avibactam can exist in the unionized form, or as a pharmaceutically acceptable salt thereof. As such, the term “avibactam” encompasses the compound in the unionized form, as well as pharmaceutically acceptable salts thereof. The structural formula is shown below.

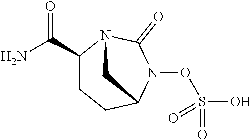

The term “EDTA” or “ethylenediaminetetraacetic acid” or “EDTA acid” as used herein refers to an aminopolycarboxylic acid with the formula [CH₂N(CH₂CO₂H)₂]₂. EDTA has the IUPAC name N,N′-(Ethane-1,2-diyl)bis[N-(carboxymethyl)glycine]; CAS number 60-00-4 (free acid), 6381-92-6 (dihydrate disodium salt). EDTA has the chemical formula C₁₀H₁₆N₂O₈ and molar mass 292.244 g·mol⁻¹. EDTA is available as several salts, notably disodium EDTA, sodium calcium edetate, and tetrasodium EDTA. As such, the term “EDTA” encompasses the compound in the unionized form, as well as pharmaceutically acceptable salts thereof. The structural formula is shown below.

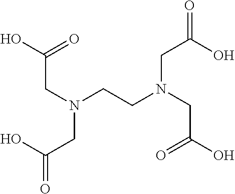 [SMILES: O=C(O)CN(CCN(CC(=O)O)CC(=O)O)CC(=O)O]

The term “combination,” “combination of actives,” or “pharmaceutical combination” as used herein refers to the combination of meropenem, one or more antibiotic resistance breakers, and optionally one or more additional antibacterial agents. In specific embodiments, the combination of actives can include meropenem, avibactam, and EDTA. The combination of actives (e.g., meropenem, one or more antibiotic resistance breakers, and optionally one or more additional antibacterial agents) may thus be administered together, e.g., in a single pharmaceutical dosage form. Alternatively, the combination of actives (e.g., meropenem, one or more antibiotic resistance breakers, and optionally one or more additional antibacterial agents) may be administered separately, e.g., they can be included within separate pharmaceutical dosage forms. The combination of actives (e.g., meropenem, one or more antibiotic resistance breakers, and optionally one or more additional antibacterial agents) may be formulated within pharmaceutical compositions that are sold independently of each other with instructions for their combined use are provided in the package equipment, e.g. leaflet or the like, or in other information e.g. provided to physicians and medical staff (e.g. oral communications, communications in writing or the like), for simultaneous or sequential use for being jointly active, especially as defined below. It can refer to either a fixed combination in one dosage unit form, or a kit of parts for the combined administration of combination of actives may be administered independently at the same time or separately within time intervals, especially where these time intervals allow that the combination of actives show a synergistic effect.

The terms “co-administration” or “combined administration” or “combined use” or the like as utilized herein are meant to encompass administration of the selected combination of actives to a single subject in need thereof (e.g. a patient), and are intended to include treatment regimens in which the actives are not necessarily administered by the same route of administration and/or at the same time.

The term “fixed combination” means that the combination of actives are both administered to a patient simultaneously in the form of a single entity or dosage. In other terms, the combination of actives can be present in one dosage form, e.g. in one tablet or in one capsule.

The term “non-fixed combination” means that the combination of actives are both administered to a patient as separate entities either simultaneously, concurrently, or sequentially with no specific time limits, wherein such administration provides therapeutically effective levels of the compounds in the body of the patient. The ratio of the total amounts of the combination active (i) to the combination active (ii) to be administered in the combined preparation can be varied, e.g. in order to cope with the needs of a patient sub-population to be treated or the needs of the single patient which different needs can be due to age, sex, body weight, etc. of the patients.

The term “pharmaceutically inert ingredient” or “inactive ingredient” or “carrier” or “excipient” refers to and includes compounds or materials used to facilitate administration of a compound (e.g., active), for example, to increase the solubility of the compound. Typical, non-limiting examples of solid carriers include starch, lactose, dicalcium phosphate, sucrose, and kaolin. Typical, non-limiting examples of liquid carriers include sterile water, saline, buffers, non-ionic surfactants, and edible oils. In addition, various adjuvants commonly used in the art may also be included. These and other such compounds are described in literature, e.g., in the Merck Index (Merck & Company, Rahway, N.J.). Considerations for inclusion of various components in pharmaceutical compositions are described, e.g., in Gilman et al. (Goodman and Gilman's: The Pharmacological Basis of Therapeutics, 8th Ed., Pergamon Press., 1990), which is incorporated herein by reference in its entirety.

The term “subject” as used herein refers to vertebrate or invertebrate, including a mammal. The term “subject” includes human, animal, a bird, a fish, or an amphibian. Typical, non-limiting examples of a “subject” include humans, cats, dogs, horses, sheep, bovine cows, pigs, lambs, rats, mice, and guinea pigs.

The term “pharmaceutically acceptable salt” as used herein refers to one or more salts of a given compound which possesses desired pharmacological activity of the free compound, and which is neither biologically nor otherwise undesirable. In general, the term “pharmaceutically acceptable salts” refer to salts that are suitable for use in contact with the tissues of human and animals without undue toxicity, irritation, allergic response and the like, and are commensurate with a reasonable benefit/risk ratio.

The present invention broadly relates to the combination of meropenem and one or more antibiotic resistance breakers, pharmaceutical compositions containing the same, and methods for treating bacterial infections that include administering the same.

The present invention specifically relates to the combination of (a) meropenem, (b) avibactam, and (c) EDTA; pharmaceutical compositions containing the same, and methods for treating bacterial infections that include administering the same.

The inventors have surprisingly found that a combination of (a) meropenem, (b) avibactam, and (c) EDTA exhibits unexpectedly improved antibacterial efficacy, even against highly resistant bacteria, including those producing extended spectrum beta-lactamase enzymes (ESBLs) and carbapenamases (Metallo Beta Lactamases).

Surprisingly, it has been found that pharmaceutical compositions comprising meropenem, avibactam, and EDTA exhibit unexpectedly synergistic antibacterial activity, even against highly resistant bacterial strains. Without wishing to be bound by any theory, it is believed that the antibiotic resistance breakers such as Avibactam and EDTA (which are beta-lactamase inhibitors) in combination with Meropenem (a Beta-lactam antibiotic) inhibit the beta-lactamase enzyme responsible for degradation of Meropenem, thereby enhancing the antimicrobial activity and helps in overcoming resistance barriers.

The present invention relates to a pharmaceutical combination comprising (a) an effective amount of meropenem, (b) an effective amount of avibactam, and (c) an effective amount of EDTA.

In an embodiment, there is provided a pharmaceutical combination comprising (a) 1 gram to 10 gram meropenem (b) 1 gram to 5 gram avibactam and (c) 100 mg to 300 mg EDTA or a pharmaceutically acceptable salt thereof.

Individual amounts of meropenem or avibactam may vary depending on clinical requirements.

In an embodiment, the combination comprises the dose in any one of the following amounts:
(i) about 3 gram of meropenem and about 1.5 gram of avibactam, 111 mg EDTA or a pharmaceutically acceptable salt; or
(ii) about 3 gram of meropenem and about 1.5 gram of avibactam 222 mg EDTA or a pharmaceutically acceptable salt; or
(iii) about 3 gram of meropenem and about 3 gram of avibactam 111 mg EDTA or a pharmaceutically acceptable salt; or
(iv) about 3 gram of meropenem and about 3 gram of avibactam 222 mg EDTA or a pharmaceutically acceptable salt; or
(v) about 6 gram of meropenem and about 1.5 gram of avibactam, 111 mg EDTA or a pharmaceutically acceptable salt; or
(vi) about 6 gram of meropenem and about 3 gram of avibactam 222 mg EDTA or a pharmaceutically acceptable salt.

In an embodiment, the EDTA is present in the range from about 0.001 mg/ml to about 15 mg/ml after reconstitution. Preferably, EDTA is present in the range from about 0.1 mg/ml to about 10 mg/ml after reconstitution. Most preferably, EDTA is present in the range from about 1 mg/ml to 5 mg/ml after reconstitution.

In an embodiment, the present invention provides a pharmaceutical composition comprising the combination of (a) meropenem, (b) avibactam, and (c) EDTA, along with one or more pharmaceutically acceptable carriers or excipients or the like.

The pharmaceutical compositions or the active ingredients according to the present invention may be formulated into a variety of dosage forms, such as solid, semi-solid, liquid and aerosol dosage forms. Typical, non-limiting examples of some dosage forms include tablets, capsules, powders, solutions, suspensions, suppositories, aerosols, granules, emulsions, syrups, elixirs, ointment, gel, cream, liposomal composition and the like.

In some embodiments, pharmaceutical compositions according to the invention are in the form of a powder or a solution. In some other embodiments, pharmaceutical compositions according to the invention are present in the form of a powder or a solution that can be reconstituted by addition of a compatible reconstitution diluent prior to administration. In some other embodiments, pharmaceutical compositions according to the invention are in the form of a frozen composition that can be diluted with a compatible reconstitution diluent prior to administration. Typical, non-limiting examples of suitable compatible reconstitution diluents include water for injection, 5% dextrose, 0.9% saline, and 5% dextrose+0.9% saline.

In some other embodiments, pharmaceutical compositions according to the invention are present in the form ready to use for parenteral administration.

The compositions according to the invention can be formulated into various dosage forms wherein the active ingredients and/or excipients may be present either together or as separate components. When the various ingredients in the composition are formulated as a mixture, such compositions can be delivered by administering such a mixture to a subject using any suitable route of administration. Alternatively, pharmaceutical compositions according to the invention may also be formulated into a dosage form wherein one or more ingredients (such as active or inactive ingredients) are present as separate components. The composition or dosage form wherein the ingredients do not come as a mixture, but come as separate components, such composition/dosage form may be administered in several ways. In one possible way, the ingredients may be mixed in the desired proportions and the mixture is reconstituted in suitable reconstitution diluent and is then administered as required. Alternatively, the components or the ingredients (active or inert) may be separately administered (simultaneously or one after the other) in appropriate proportion so as to achieve the same or equivalent therapeutic level or effect as would have been achieved by administration of the equivalent mixture.

In some embodiments, pharmaceutical compositions according to the invention are formulated into a dosage form such that meropenem, avibactam, and EDTA are present in the composition together or as separate components. In some other embodiments, pharmaceutical compositions according to the invention are formulated into a dosage form such that meropenem, avibactam, and EDTA are present in the composition as separate components.

In another embodiment of the present invention, the required amount of the pharmaceutical composition is provided in a sealed airtight container such as a vial, an ampoule, a syringe, a packet, a pouch or an auto-injector. These containers can contain the compositions disclosed in this invention in volumes of a single dose or in volumes of multiple doses up to 10. The interior space of the sealed airtight container comprises a fill volume occupied by the formulation of the present invention and a headspace volume occupied aseptically by an inert-gas-limited micro-atmosphere, which micro-atmosphere comprises essentially of one or more inert gases selected from the group consisting of noble gases and nitrogen, such that the ratio of the fill volume to headspace volume is not less than 1:1.

The pharmaceutically effective dosage of the composition, in the form of the concentrate of the dose, can be provided in a sealed airtight container, wherein the container has a head space volume sufficient for introduction of appropriate volume of an aqueous solvent/compatible diluent selected from a group of sterile water for injection, bacteriostatic water for injection and isotonic sterile sodium chloride solution sufficient to form an appropriate reconstituted solution of the composition.

In case of unit/multiple dose of the composition, the pharmaceutically effective dose can be provided in a sealed airtight container, wherein the container has a head space volume sufficient for introduction of appropriate volume of an aqueous solvent. Unit/multiple dose is in the form of an appropriate reconstituted solution of the composition.

For use as an injection, the composition can be provided in the form of a sterile dry powder, in a sealed airtight container, to form a pharmaceutically acceptable required fixed dose combination for reconstitution prior to intramuscular or intravenous administration for the treatment of the bacterial infections.

As another alternative, the composition can be provided in a sealed container such as transparent glass vial capped with appropriate halogenated stopper and seal and is used for reconstitution for intramuscular or intravenous administration for the treatment of the bacterial infections. When the composition is provided in a reconstituted form in a sealed airtight container, the interior space of the container can comprise a fill volume occupied by the composition in reconstituted form and a head space volume occupied aseptically by an inert-gas-limited micro atmosphere, which can comprise essentially one or more inert gas as selected from the group consisting of noble gases and nitrogen, preferably nitrogen, volume of the nitrogen gas being not more than 5% of the head space volume, and wherein ratio of said fill volume to the head space volume is not less than 1:1.

In an embodiment, pharmaceutical compositions according to the invention are used in treatment of bacterial infection.

In another embodiment, there are provided methods for treating bacterial infection in a subject, said method comprising administering to said subject effective amount of a pharmaceutical composition according to the invention. In case of dosage forms wherein meropenem, avibactam, and EDTA are present in the composition as separate components; meropenem may be administered before, after, or simultaneously with the administration of avibactam along with EDTA.

In yet another embodiment, there are provided methods for treating bacterial infections in a subject, said methods comprising administering to said subject an effective amount of (a) meropenem, (b) avibactam, and (c) EDTA.

In another embodiment, there are provided methods for treating bacterial infections in a subject, said methods comprising administering to said subject a daily dose of (a) 1 gram to 10 gram meropenem, (b) 1 gram to 5 gram avibactam, and (c) 100 mg to 300 mg EDTA.

In another embodiment, there are provided methods for treating bacterial infections in a subject, said methods comprising administering the said daily dose to the subject at an interval of 8 hours.

In some embodiments, there is provided a method for treating a bacterial infection in a subject, said method comprising administering to said subject a daily dose of: (a) meropenem, (b) avibactam, and (c) EDTA in any of the following amounts:
(i) about 3 gram of meropenem and about 1.5 gram of avibactam, 111 mg EDTA or a pharmaceutically acceptable salt or
(ii) about 3 gram of meropenem and about 1.5 gram of avibactam 222 mg EDTA or a pharmaceutically acceptable salt; or
(iii) about 3 gram of meropenem and about 3 gram of avibactam 111 mg EDTA or a pharmaceutically acceptable salt; or
(iv) about 3 gram of meropenem and about 3 gram of avibactam 222 mg EDTA or a pharmaceutically acceptable salt; or
(v) about 6 gram of meropenem and about 1.5 gram of avibactam, 111 mg EDTA or a pharmaceutically acceptable salt; or
(vi) about 6 gram of meropenem and about 3 gram of avibactam 222 mg EDTA or a pharmaceutically acceptable salt.

In some embodiments, there is provided a method for treating a bacterial infection in pediatric patients, said method comprising administering to said subject a daily dose of (a) meropenem from about 10 mg/kg to about 40 mg/kg, (b) avibactam from about 10 mg/kg to about 12.5 mg/kg, and (c) EDTA from about 1.85 mg/kg to about 3.7 mg/kg.

In yet another embodiment, in the method according to the invention, meropenem is administered before, after, or simultaneously with the administration of avibactam, EDTA.

In yet another embodiment, in the method according to the invention the daily dose of (a) meropenem, (b) avibactam, and (c) EDTA is administered at an interval of 8 hours.

In yet another embodiment, in the method according to the invention (a) meropenem, (b) avibactam, and (c) EDTA is administered as an intravenous infusion from about 30 minutes to about 120 minutes.

In the methods according to the invention, the pharmaceutical composition and/or other pharmaceutically active ingredients disclosed herein may be administered by any appropriate method, which serves to deliver the composition, or its constituents, or the active ingredients to the desired site. The method of administration can vary depending on various factors, such as for example, the components of the pharmaceutical composition and the nature of the active ingredients, the site of the potential or actual infection, the microorganism (e.g. bacteria) involved, severity of infection, age, and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In some embodiments, the compositions or one or more active ingredients according to the invention are administered parenterally.

In one embodiment, there is provided a method for increasing antibacterial effectiveness in a subject, said method comprising co-administering meropenem, avibactam, and EDTA. In other embodiments, there is provided a method for increasing antibacterial effectiveness in a subject, said method comprising co-administering meropenem, avibactam, and EDTA.

A wide variety of bacterial infections can be treated using compositions and methods according to the invention.

The pharmaceutical compositions and methods according to the invention are useful in treatment or prevention of several infections, including for example, urinary tract infections, complicated urinary tract infections, respiratory tract infections, pneumonia, surgical infections, intraabdominal infections, skin and soft tissue infections, blood stream infections and the like.

In one embodiment, pharmaceutical compositions and methods according to the invention are used in treatment or prevention of infections caused by resistant bacteria. In other embodiment, the compositions and methods according to the invention are used in treatment or prevention of infections caused by bacteria producing one or more beta-lactamase enzymes.

In general, the pharmaceutical compositions and methods disclosed herein are also effective in treating infections caused by bacteria that are considered to be less or not susceptible to one or more of known antibacterial agents or their known compositions. Some non-limiting examples of such bacteria known to have developed resistance to various antibacterial agents include Acinetobacter, Escherichia coli, Pseudomonas aeruginosa, Staphylococcus aureus, Enterobacter, Klebsiella, Citrobacter and the like.

### Enumerated Embodiments

Specific enumerated embodiments <1> to <75> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Embodiment <1>

A composition comprising meropenem and one or more antibiotic resistance breakers.

### Embodiment <2>

The composition of embodiment <1> in a bulk form.

### Embodiment <3>

The composition of embodiment <1>, which is a pharmaceutical composition, further comprising one or more pharmaceutically acceptable excipients.

### Embodiment <4>

The composition of embodiment <1>, which is a pharmaceutical composition in a unit dosage form, further comprising one or more pharmaceutically acceptable excipients.

### Embodiment <5>

The composition of any one of embodiments <1> to <4>, wherein the one or more antibiotic resistance breakers comprises avibactam.

### Embodiment <6>

The composition of any one of embodiments <1> to <4>, wherein the one or more antibiotic resistance breakers comprises 1-5 grams avibactam.

### Embodiment <7>

The composition of any one of embodiments <1> to <4>, wherein the one or more antibiotic resistance breakers comprises 1.5-3 grams avibactam.

### Embodiment <8>

The composition of any one of embodiments <1> to <4>, wherein the one or more antibiotic resistance breakers comprises 3±1 grams avibactam.

### Embodiment <9>

The composition of any one of embodiments <1> to <4>, wherein the one or more antibiotic resistance breakers comprises 3±0.5 grams avibactam.

### Embodiment <10>

The composition of any one of embodiments <1> to <4>, wherein the one or more antibiotic resistance breakers comprises 3 grams avibactam.

### Embodiment <11>

The composition of any one of embodiments <1> to <4>, wherein the one or more antibiotic resistance breakers comprises 1.5±0.5 grams avibactam.

### Embodiment <12>

The composition of any one of embodiments <1> to <4>, wherein the one or more antibiotic resistance breakers comprises 1.5±0.25 grams avibactam.

### Embodiment <13>

The composition of any one of embodiments <1> to <4>, wherein the one or more antibiotic resistance breakers comprises 1.5 grams avibactam.

### Embodiment <14>

The composition of any one of embodiments <1> to <13>, wherein the one or more antibiotic resistance breakers comprises EDTA.

### Embodiment <15>

The composition of any one of embodiments <1> to <13>, wherein the one or more antibiotic resistance breakers comprises 100-300 mg EDTA.

### Embodiment <16>

The composition of any one of embodiments <1> to <13>, wherein the one or more antibiotic resistance breakers comprises 110-225 mg EDTA.

### Embodiment <17>

The composition of any one of embodiments <1> to <13>, wherein the one or more antibiotic resistance breakers comprises 222±20 mg EDTA.

### Embodiment <18>

The composition of any one of embodiments <1> to <13>, wherein the one or more antibiotic resistance breakers comprises 222±10 mg EDTA.

### Embodiment <19>

The composition of any one of embodiments <1> to <13>, wherein the one or more antibiotic resistance breakers comprises 222±5 mg EDTA.

### Embodiment <20>

The composition of any one of embodiments <1> to <13>, wherein the one or more antibiotic resistance breakers comprises 222 mg EDTA.

### Embodiment <21>

The composition of any one of embodiments <1> to <13>, wherein the one or more antibiotic resistance breakers comprises 111±10 mg EDTA.

### Embodiment <22>

The composition of any one of embodiments <1> to <13>, wherein the one or more antibiotic resistance breakers comprises 111±5 mg EDTA.

### Embodiment <23>

The composition of any one of embodiments <1> to <13>, wherein the one or more antibiotic resistance breakers comprises 111±2.5 mg EDTA.

### Embodiment <24>

The composition of any one of embodiments <1> to <13>, wherein the one or more antibiotic resistance breakers comprises 111 mg EDTA.

### Embodiment <25>

The composition of any one of embodiments <1> to <13>, wherein the one or more antibiotic resistance breakers comprises avibactam and EDTA.

### Embodiment <26>

The composition of any one of embodiments <1> to <13>, wherein the one or more antibiotic resistance breakers comprises 1-5 grams avibactam and 100-300 mg EDTA.

### Embodiment <27>

The composition of any one of embodiments <1> to <13>, wherein the one or more antibiotic resistance breakers comprises 1.5-3 grams avibactam and 110-225 mg EDTA.

### Embodiment <28>

The composition of any one of embodiments <1> to <13>, wherein the one or more antibiotic resistance breakers comprises 1.5±0.5 grams avibactam and 111+10 mg EDTA.

### Embodiment <29>

The composition of any one of embodiments <1> to <13>, wherein the one or more antibiotic resistance breakers comprises 1.5±0.25 grams avibactam and 111±5 mg EDTA.

### Embodiment <30>

The composition of any one of embodiments <1> to <13>, wherein the one or more antibiotic resistance breakers comprises 1.5±0.15 grams avibactam and 111±5 mg EDTA.

### Embodiment <31>

The composition of any one of embodiments <1> to <30>, wherein the meropenem is present in 1-10 grams.

### Embodiment <32>

The composition of any one of embodiments <1> to <30>, wherein the meropenem is present in 3-6 grams.

### Embodiment <33>

The composition of any one of embodiments <1> to <30>, wherein the meropenem is present in 6±1 grams.

### Embodiment <34>

The composition of any one of embodiments <1> to <30>, wherein the meropenem is present in 6±0.5 grams.

### Embodiment <35>

The composition of any one of embodiments <1> to <30>, wherein the meropenem is present in 6±0.25 grams.

### Embodiment <36>

The composition of any one of embodiments <1> to <30>, wherein the meropenem is present in 6 grams.

### Embodiment <37>

The composition of any one of embodiments <1> to <30>, wherein the meropenem is present in 3±0.5 grams.

### Embodiment <38>

The composition of any one of embodiments <1> to <30>, wherein the meropenem is present in 3±0.25 grams.

### Embodiment <39>

The composition of any one of embodiments <1> to <30>, wherein the meropenem is present in 3±0.15 grams.

### Embodiment <40>

The composition of any one of embodiments <1> to <30>, wherein the meropenem is present in 3 grams.

### Embodiment <41>

The composition of any one of embodiments <1> to <40>, which is in the form of a unit dosage form selected from the group consisting of oral tablet, oral capsule, powder, oral solution, oral suspension, suppository, aerosol, granule, emulsion, syrup, elixir, ointment, gel, cream, liquid injectable, and liposomal composition.

### Embodiment <42>

The composition of any one of embodiments <1> to <40>, which is in the form of a unit dosage form and formulated for delivery to a human subject via oral, intravenous, topical, intrarespiratory, intraperitoneal, intramuscular, parenteral, sublingual, transdermal, intranasal, pulmonary, intraocular, intratracheal, intrarectal, or vaginal.

### Embodiment <43>

The composition of any one of embodiments <1> to <40>, which is in the form of a dry powder, suitable for reconstitution in a liquid carrier for parenteral administration.

### Embodiment <44>

The composition of any one of embodiments <1> to <40>, which is in the form of a dry powder, suitable for reconstitution in a liquid carrier for parenteral administration, the composition further comprising one or more pharmaceutical acceptable excipients in dry form.

### Embodiment <45>

The composition of any one of embodiments <1> to <40>, which is in the form of a dry powder, suitable for reconstitution in a liquid carrier for parenteral administration, the composition further comprising one or more pharmaceutical acceptable excipients in dry form, the one or more pharmaceutical acceptable excipients comprise at least one of a buffer, pH adjusting agent, antioxidant, and preservative.

### Embodiment <46>

The composition of any one of embodiments <1> to <40>, which is in the form of a dry powder unit dosage form contained within a vial, the composition is suitable for reconstitution in a liquid carrier for parenteral administration.

### Embodiment <47>

The composition of any one of embodiments <1> to <40>, which is in the form of a dry powder unit dosage form contained within a glass vial, the composition is suitable for reconstitution in a liquid carrier for parenteral administration.

### Embodiment <48>

The composition of any one of embodiments <1> to <40>, which is in the form of a dry powder, suitable for reconstitution in a liquid carrier, for parenteral administration, wherein the liquid carrier comprises at least one of water for injection, saline, and dextrose.

### Embodiment <49>

The composition of any one of embodiments <1> to <40>, which is in the form of a liquid injectable, suitable for parenteral administration, wherein the composition comprises a liquid carrier comprising at least one of water for injection, saline, and dextrose.

### Embodiment <50>

The composition of any one of embodiments <1> to <40>, which is in the form of a liquid injectable, suitable for parenteral administration, wherein the composition comprises a liquid carrier comprising at least one of water for injection, 0.9% saline, and 5% dextrose.

### Embodiment <51>

The composition of any one of embodiments <43> to <50>, which is administered parenterally.

### Embodiment <52>

The composition of any one of embodiments <43> to <50>, which is administered parenterally at an interval of 8 hours.

### Embodiment <53>

The composition of any one of embodiments <43> to <50>, which is administered as an intravenous infusion.

### Embodiment <54>

The composition of any one of embodiments <43> to <50>, which is administered as an intravenous infusion from about 30 minutes to about 120 minutes.

### Embodiment <55>

A method of treating an infection, the method comprising administering to a human subject afflicted with an infection, or at risk thereof, with the pharmaceutical composition of any one of embodiments <1> to <54>, effective to treat the infection.

### Embodiment <56>

The method of embodiment <55>, wherein the infection is a bacterial infection.

### Embodiment <57>

The method of embodiment <55>, wherein the infection is a bacterial infection caused by resistant bacteria.

### Embodiment <58>

The method of embodiment <55>, wherein the infection is a bacterial infection caused by bacteria producing one or more beta-lactamase enzymes.

### Embodiment <59>

The method of embodiment <55>, wherein the infection is a bacterial infection caused by resistant bacteria comprising at least one of Acinetobacter, Escherichia coli, Pseudomonas aeruginosa, Staphylococcus aureus, Enterobacter, Klebsiella, and Citrobacter.

### Embodiment <60>

The method of any one of embodiments <55> to <59>, wherein the infection is a urinary tract infection, complicated urinary tract infection, respiratory tract infection, pneumonia, surgical infection, intraabdominal infection, skin and soft tissue infection, or blood stream infections.

### Embodiment <61>

The method of any one of embodiments <55> to <60>, wherein the administration of the pharmaceutical composition is an intravenous infusion and results in a daily dose to the human subject of (a) meropenem from about 10 mg/kg to about 40 mg/kg, (b) avibactam from about 10 mg/kg to about 12.5 mg/kg, and (c) EDTA from about 1.85 mg/kg to about 3.7 mg/kg.

### Embodiment <62>

The method of any one of embodiments <55> to <60>, wherein the administration of the pharmaceutical composition results in a daily dose to the human subject of (a) meropenem in about 25±10 mg/kg, (b) avibactam in about 11.25±4 mg/kg, and (c) EDTA in 2.775±0.6 mg/kg.

### Embodiment <63>

The method of any one of embodiments <55> to <60>, wherein the administration of the pharmaceutical composition results in a daily dose to the human subject of (a) meropenem in about 25±5 mg/kg, (b) avibactam in about 11.25±2 mg/kg, and (c) EDTA in 2.775±0.3 mg/kg.

### Embodiment <64>

The method of any one of embodiments <55> to <60>, wherein the administration of the pharmaceutical composition results in a daily dose to the human subject of (a) meropenem in about 25±2.5 mg/kg, (b) avibactam in about 11.25±1 mg/kg, and (c) EDTA in 2.775±0.15 mg/kg.

### Embodiment <65>

The method of any one of embodiments <55> to <60>, wherein the administration of the pharmaceutical composition results in a daily dose to the human subject of (a) meropenem from about 10 mg/kg to about 40 mg/kg, (b) avibactam from about 10 mg/kg to about 12.5 mg/kg, and (c) EDTA from about 1.85 mg/kg to about 3.7 mg/kg.

### Embodiment <66>

The method of any one of embodiments <55> to <60>, wherein the administration of the pharmaceutical composition is an intravenous infusion and results in a daily dose to the human subject of (a) meropenem in about 25±5 mg/kg, (b) avibactam in about 11.25±2.2 mg/kg, and (c) EDTA in about 2.275±0.5 mg/kg.

### Embodiment <67>

The method of any one of embodiments <55> to <60>, wherein the administration of the pharmaceutical composition is an intravenous infusion and results in a daily dose to the human subject of (a) meropenem in about 25±2.5 mg/kg, (b) avibactam in about 11.25±1.1 mg/kg, and (c) EDTA in about 2.275±0.25 mg/kg.

### Embodiment <68>

The method of any one of embodiments <55> to <60>, wherein the administration of the pharmaceutical composition is an intravenous infusion and results in a daily dose to the human subject of (a) meropenem in about 25±1.5 mg/kg, (b) avibactam in about 11.25±0.5 mg/kg, and (c) EDTA in about 2.275±0.15 mg/kg.

### Embodiment <69>

The method of any one of embodiments <55> to <68>, wherein the meropenem is administered simultaneously with the administration of avibactam and EDTA.

### Embodiment <70>

The method of any one of embodiments <55> to <68>, wherein the meropenem is administered before the administration of avibactam and EDTA.

### Embodiment <71>

The method of any one of embodiments <55> to <68>, wherein the meropenem is administered after the administration of avibactam and EDTA.

### Embodiment <72>

The method of any one of embodiments <55> to <68>, wherein the meropenem, avibactam, and EDTA are present within the same dosage form.

### Embodiment <73>

The method of any one of embodiments <55> to <68>, wherein the meropenem, avibactam, and EDTA are not present within the same dosage form.

### Embodiment <74>

The method of any one of embodiments <55> to <68>, wherein the meropenem is present within a first dosage form and the avibactam and EDTA are present within a second dosage form.

### Embodiment <75>

The method of any one of embodiments <55> to <68>, wherein the meropenem is present within a first dosage form, the avibactam is present within a second dosage form, and the EDTA is present within a third dosage form.

### EXAMPLES

The following examples are meant to illustrate the present invention. The examples are presented to exemplify the invention and are not to be considered as limiting the scope of the invention. The antibacterial activity of combinations according to the invention was investigated against various resistant bacterial strains.

In a typical study MIC tests were performed by broth microdilution in line with CLSI susceptibility testing standards. Bacterial inocula were prepared at ca. 1×10⁶ CFU/mL by diluting 100-fold a 0.5 McFarland suspension in CA-MHB with TES. Microtiter plate wells containing 50 μL of antibacterial solutions at 2×the final concentrations were diluted 2-fold with 50 μL of inoculum to give a final inoculum of ca. 5×10⁵ CFU/mL and desired test concentrations of antibacterial agents. Test microtiter plates were incubated according to CLSI guidelines and read visually. MIC values corresponded to the first well with no visible growth.

### Example 1

Against all grouped isolates from India (606), the activity of the Amikacin (AMK) and Ceftazidime avibactam (CZA) was low with MIC₉₀ values of >64 or >32 μg/mL. The Meropenem (MEM) MIC₅₀ and MIC₉₀ values were >32 μg/mL. There was a marked improvement in antibacterial activity, compared to MEM alone, observed for the triple combinations, for MEM/AVI [4 mg/L]/EDTA [10 mM] and MEM/AVI [4 mg/L]/EDTA [20 mM], the reduction in MIC₉₀ was 0.06 μg/mL and ≤0.002 μg/mL, respectively. The MEM/AVI [8 mg/L]/EDTA [10 mM] and MEM/AVI [8 mg/L]/EDTA [20 mM] displayed a significant improvement in MIC₉₀ to ≤0.002 μg/mL compared to MEM alone.

The results are shown in table 1.

| Drug | MIC Range (μg/mL) | MIC50 | MIC90 |
|---|---|---|---|
| Amikacin | ≤2->64 | >64 | >64 |
| Ceftazidime-avibactam | ≤0.12->32 | >32 | >32 |
| Ceftriaxone with sulbactam at 4 | ≤0.03->64 | ≤0.03 | 2 |
| and EDTA at 10 mM | | | |
| Meropenem | ≤0.015->32 | >32 | >32 |
| Meropenem with AVI at 4 and | ≤0.002-0.5 | ≤0.002 | 0.06 |
| EDTA at 10 mM | | | |
| Meropenem with AVI at 4 and | ≤0.002-1 | ≤0.002 | ≤0.002 |
| EDTA at 20 mM | | | |
| Meropenem with AVI at 8 and | ≤0.002-0.25 | ≤0.002 | ≤0.002 |
| EDTA at 10 mM | | | |
| Meropenem with AVI at 8 and | ≤0.002-0.5 | ≤0.002 | ≤0.002 |
| EDTA at 20 mM | | | |

Against MEM-NS (Meropenem nonsusceptible-85.9%) grouped isolates (n=534), MEM alone showed low antibacterial activity with MIC₅₀ and MIC₉₀>32 μg/mL, respectively, and a MIC range between 2 and >32 μg/mL. MEM/AVI [4 mg/L]/EDTA [10 mM] and MEM/AVI [4 mg/L]/EDTA [20 mM] had MIC₉₀ values of 0.06 and ≤0.002 μg/mL, respectively. MEM/A VI [8 mg/L]/EDTA [10 mM] and MEM/AVI [8 mg/L]/EDTA [20 mM] had MIC₉₀ values of ≤0.002 μg/mL.

### Example 2

The results of antibacterial activity of amikacin, Ceftazidime-avibactam, Ceftriaxone with sulbactam and EDTA, Meropenem and Meropenem with avibactam and EDTA against highly resistant strains of Enterobacteriaceae are given in Table 2. As may be seen, against against all grouped Enterobacterales isolates from India (n=301), the activity of the comparator compounds was low, AMK and CZA displays MIC₉₀ of >64 and >32 μg/mL, for CRO/S4/E10 MIC₉₀ was 16 μg/mL.

MEM MIC₅₀ and MIC₉₀ were at >32 μg/mL (MIC range: ≤0.015->32 μg/mL) and 250 out of 301 (82.7%) (MEM NS) isolates were resistant to meropenem. When combined with AVI [4 mg/L] and the addition of EDTA at 10 or 20 mM reduced the MIC₉₀ to 0.125 and ≤0.002 μg/mL, respectively. Similarly with AVI [8 mg/L] the MIC₉₀ for EDTA at 10 or 20 mM improved to 0.015 and ≤0.002 μg/mL, respectively.

| Drug | MIC Range (μg/mL) | MIC50 | MIC90 |
|---|---|---|---|
| Amikacin | ≤2->64 | >64 | >64 |
| Ceftazidime-avibactam | ≤0.12->32 | >32 | >32 |
| Ceftriaxone with sulbactam at 4 | ≤0.03->64 | ≤0.03 | 16 |
| and EDTA at 10 mM | | | |
| Meropenem | ≤0.015->32 | >32 | >32 |
| Meropenem with AVI at 4 and | ≤0.002-0.5 | ≤0.002 | 0.12 |
| EDTA at 10 mM | | | |
| Meropenem with AVI at 4 and | ≤0.002-1 | ≤0.002 | ≤0.002 |
| EDTA at 20 mM | | | |
| Meropenem with AVI at 8 and | ≤0.002-0.25 | ≤0.002 | 0.015 |
| EDTA at 10 mM | | | |
| Meropenem with AVI at 8 and | ≤0.002-0.5 | ≤0.002 | ≤0.002 |
| EDTA at 20 mM | | | |

5 Against MEM-NS grouped Enterobacterales isolates (n=250, 82.7%), MEM alone showed low antibacterial activity with MIC₅₀ and MIC₉₀ at >32 μg/mL and a MIC range between 2 and >32 μg/mL. MEM/AVI [4 mg/L]/EDTA [10 mM] and MEM/AVI [4 mg/L]/EDTA [20 mM] had considerably lower MIC₉₀ values of 0.12, and 0.004 μg/mL, respectively.

### Example 3

The results of antibacterial activity of amikacin, Ceftazidime-avibactam, Ceftriaxone with sulbactam and EDTA, Meropenem and Meropenem with avibactam and EDTA against highly resistant strains of Escherichia coli are given in Table 3. Against E. coli, the MEM MIC₅₀ and MIC₉₀ was >32 μg/mL. There was a marked improvement in antibacterial activity, compared to MEM alone, observed for the triple combination MEM/AVI [4 mg/L]/EDTA [10 mM] with a MIC₉₀ at 0.004 μg/mL

| Drug | MIC Range (μg/mL) | MIC50 | MIC90 |
|---|---|---|---|
| Amikacin | ≤2->64 | 8 | >64 |
| Ceftazidime-avibactam | ≤0.12->32 | >32 | >32 |
| Ceftriaxone with sulbactam at 4 | ≤0.03->64 | ≤0.03 | 1 |
| and EDTA at 10 mM | | | |
| Meropenem | ≤0.015->32 | >32 | >32 |
| Meropenem with AVI at 4 and | ≤0.002-0.06 | ≤0.002 | 0.004 |
| EDTA at 10 mM | | | |
| Meropenem with AVI at 4 and | ≤0.002-0.015 | ≤0.002 | ≤0.002 |
| EDTA at 20 mM | | | |
| Meropenem with AVI at 8 and | ≤0.002-0.015 | ≤0.002 | ≤0.002 |
| EDTA at 10 mM | | | |
| Meropenem with AVI at 8 and | ≤0.002-0.008 | ≤0.002 | ≤0.002 |
| EDTA at 20 mM | | | |

Against MEM-NS E. coli isolates (n=105, 88.2%,), MEM MIC₅₀ and MIC₉₀ was >32 μg/mL. However, MEM/AVI [4 mg/L]/EDTA [10 mM] and MEM/AVI [4 mg/L]/EDTA [20 mM] had MIC₉₀ values of 0.004 and ≤0.002 μg/mL, respectively which are considerably lower.

### Example 4

The results of antibacterial activity of amikacin, Ceftazidime-avibactam, Ceftriaxone with sulbactam and EDTA, Meropenem and Meropenem with avibactam and EDTA against highly resistant strains of Klebsiella (93. 3% Mem NS) are given in Table 4. As may be seen, MEM alone showed MIC₉₀ of >32 μg/mL against K. pneumoniae. The activity of AMK and CZA was much lower with MIC₉₀ of >64 and >32 μg/mL, respectively and for CRO/S4/E10 MIC₉₀ was 64 μg/mL.

5 The combinations of MEM/AVI [4 mg/L] with EDTA at 10, or 20 mM showed higher activity, resulting in MIC 90 of 0.25 and 0.12 μg/mL, respectively. Whereas MEM/AVI [8 mg/L] with EDTA at 10 or 20 mM further improved the MIC₉₀ to 0.06 and ≤0.002 μg/mL, respectively.

| Drug | MIC Range (μg/mL) | MIC50 | MIC90 |
|---|---|---|---|
| Amikacin | ≤2->64 | >64 | >64 |
| Ceftazidime-avibactam | ≤0.12->32 | >32 | >32 |
| Ceftriaxone with sulbactam at 4 | ≤0.03->64 | 1 | 64 |
| and EDTA at 10 mM | | | |
| Meropenem | ≤0.015->32 | >32 | >32 |
| Meropenem with AVI at 4 and | ≤0.002-0.5 | 0.015 | 0.25 |
| EDTA at 10 mM | | | |
| Meropenem with AVI at 4 and | ≤0.002-1 | ≤0.002 | 0.12 |
| EDTA at 20 mM | | | |
| Meropenem with AVI at 8 and | ≤0.002-0.25 | ≤0.002 | 0.06 |
| EDTA at 10 mM | | | |
| Meropenem with AVI at 8 and | ≤0.002-0.5 | ≤0.002 | ≤0.002 |
| EDTA at 20 mM | | | |

Against MEM-NS K. pneumoniae isolates (n=112, 93.3%), MEM alone showed low antibacterial activity with MIC₅₀ and MIC₉₀ of >32 μg/mL and a MIC range between 4 and >32 μg/mL. MEM/AVI [4 mg/L]/EDTA [10 mM] and MEM/AVI [4 mg/L]/EDTA [20 mM] showed MIC₉₀ values of 0.25 and 0.12 μg/mL, respectively.

### Example 5

The results of antibacterial activity of amikacin, Ceftazidime-avibactam, Ceftriaxone with sulbactam and EDTA, Meropenem and Meropenem with avibactum and EDTA against highly resistant strains of Non-Enterobacteriaceae are given in Table 5 As may be seen The activity of the comparator compounds was low AMK and CZA, the MIC₉₀ were >64 μg/mL and >32 μg/mL, respectively except for CRO/S4/E10 low MIC₉₀≤0.03 μg/mL was obtained.

MEM MIC₅₀ and MIC₉₀ was >32 μg/mL However when combined with AVI [4 mg/L/8 μg/mL], and EDTA at 10, or 20 mM, the MIC₉₀ value was further improved significantly to ≤0.002 μg/mL.

| Drug | MIC Range (μg/mL) | MIC50 | MIC90 |
|---|---|---|---|
| Amikacin | ≤2->64 | >64 | >64 |
| Ceftazidime-avibactam | ≤0.12->32 | >32 | >32 |
| Ceftriaxone with sulbactam at 4 | ≤0.03-2 | ≤0.03 | ≤0.03 |
| and EDTA at 10 mM | | | |
| Meropenem | ≤0.015->32 | >32 | >32 |
| Meropenem with AVI at 4 and | ≤0.002-0.5 | ≤0.002 | ≤0.002 |
| EDTA at 10 mM | | | |
| Meropenem with AVI at 4 and | ≤0.002-≤0.002 | ≤0.002 | ≤0.002 |
| EDTA at 20 mM | | | |
| Meropenem with AVI at 8 and | ≤0.002-0.5 | ≤0.002 | ≤0.002 |
| EDTA at 10 mM | | | |
| Meropenem with AVI at 8 and | ≤0.002-≤0.002 | ≤0.002 | ≤0.002 |
| EDTA at 20 mM | | | |

Against MEM-NS non-fermenter isolates (n=284, 88.9%), MEM alone showed low antibacterial activity with MIC₉₀>32 μg/mL and a MIC range between 4 and >32 μg/mL. MEM/AVI [4 and 8 mg/L]/EDTA [10 mM] and MEM/AVI [4 and 8 mg/L]/EDTA [20 mM] showed MIC₉₀ values of ≤0.002 μg/mL.

### Example 6

The results of antibacterial activity of amikacin, Ceftazidime-avibactam, Ceftriaxone with sulbactam and EDTA, Meropenem and Meropenem with avibactum and EDTA against highly resistant strains of P. aeruginosa are given in Table 5. As may be seen, the activity of the comparators was much lower although CRO/S4/E10 maximum MIC₉₀ were much lower at ≤0.03 μg/mL. MEM alone showed MIC₉₀ of >32 μg/mL However the combinations of MEM or MEM/AVI [4 and 8 mg/L] with EDTA at 10 or 20 mM led to marked improvement in the MIC₉₀ to ≤0.002 μg/mL.

| Drug | MIC Range (μg/mL) | MIC50 | MIC90 |
|---|---|---|---|
| Amikacin | ≤2->64 | >64 | >64 |
| Ceftazidime-avibactam | ≤0.12->32 | >32 | >32 |
| Ceftriaxone with sulbactam at 4 | ≤0.03-2 | ≤0.03 | ≤0.03 |
| and EDTA at 10 mM | | | |
| Meropenem | 0.06->32 | >32 | >32 |
| Meropenem with AVI at 4 and | ≤0.002-≤0.002 | ≤0.002 | ≤0.002 |
| EDTA at 10 mM | | | |
| Meropenem with AVI at 4 and | ≤0.002-≤0.002 | ≤0.002 | ≤0.002 |
| EDTA at 20 mM | | | |
| Meropenem with AVI at 8 and | ≤0.002-≤0.002 | ≤0.002 | ≤0.002 |
| EDTA at 10 mM | | | |
| Meropenem with AVI at 8 and | ≤0.002-≤0.002 | ≤0.002 | ≤0.002 |
| EDTA at 20 mM | | | |

Against MEM-NS P. aeruginosa (n=120, 92.7%), MEM alone showed low antibacterial activity and MIC₉₀ was >32 μg/mL and a MIC range between 4 and >32 μg/mL. All combinations tested showed significant reduction in the MICs, MEM/AVI [4 and 8 mg/L]/EDTA [10 mM] and MEM/AVI [4 and 8 mg/L]/EDTA [20 mM] showed significantly lower MIC₉₀ of ≤0.002 μg/mL.

### Example 7

The results of antibacterial activity of amikacin, Ceftazidime-avibactam, Ceftriaxone with sulbactam and EDTA, Meropenem and Meropenem with avibactum and EDTA against highly resistant strains of Acinetobacter baumannii are given in Table 7. As may be seen the activity of the comparators AMK and CZA the MIC₉₀ was higher >64 and >32 μg/mL although CRO/S4/E10 MIC₉₀ was lower at ≤0.002 μg/mL. MEM alone showed MIC₉₀ of >32 μg/mL. However, the combinations of MEM or MEM/AVI [4 and 8 mg/L] with EDTA at 10, or 20 mM reduced the MIC₉₀ significantly to ≤0.002 μg/mL.

| Drug | MIC Range (μg/mL) | MIC50 | MIC90 |
|---|---|---|---|
| Amikacin | ≤2->64 | >64 | >64 |
| Ceftazidime-avibactam | ≤0.12->32 | >32 | >32 |
| Ceftriaxone with sulbactam at 4 | ≤0.03-≤0.03 | ≤0.03 | ≤0.03 |
| and EDTA at 10 mM | | | |
| Meropenem | ≤0.015->32 | >32 | >32 |
| Meropenem with AVI at 4 and | ≤0.002-≤0.002 | ≤0.002 | ≤0.002 |
| EDTA at 10 mM | | | |
| Meropenem with AVI at 4 and | ≤0.002-≤0.002 | ≤0.002 | ≤0.002 |
| EDTA at 20 mM | | | |
| Meropenem with AVI at 8 and | ≤0.002-≤0.002 | ≤0.002 | ≤0.002 |
| EDTA at 10 mM | | | |
| Meropenem with AVI at 8 and | ≤0.002-≤0.002 | ≤0.002 | ≤0.002 |
| EDTA at 20 mM | | | |

Against MEM-NS A. baumannii isolates (n=163, 98.2%), MEM alone showed low antibacterial activity with MIC₉₀ of >32 μg/mL, and a MIC range between 32 and >32 μg/mL whereas MEM/AVI [4 and 8 mg/L]/EDTA [10 and 20 mM] had significantly lower MIC₉₀ values of ≤0.002 μg/mL.

### Example 8

The results of antibacterial activity of amikacin, Ceftazidime-avibactam, Ceftriaxone with sulbactam and EDTA, Meropenem and Meropenem with avibactum and EDTA against highly resistant strains of Stenotrophomonas maltophilia are given in Table 8. As may be seen MEM alone showed MIC₅₀ and MIC₉₀ of >32 μg/mL. The combinations of MEM/AVI [4/8 mg/L]/EDTA [10/20 mM] had MIC₉₀ of ≤0.002 μg/mL which is significantly lower.

| Drug | MIC Range (μg/mL) | MIC50 | MIC90 |
|---|---|---|---|
| Amikacin | >64->64 | >64 | >64 |
| Ceftazidime-avibactam | 4->32 | 32 | >32 |
| Ceftriaxone with sulbactam at 4 | ≤0.03-≤0.03 | ≤0.03 | ≤0.03 |
| and EDTA at 10 mM | | | |
| Meropenem | >32->32 | >32 | >32 |
| Meropenem with AVI at 4 and | ≤0.002-≤0.002 | ≤0.002 | ≤0.002 |
| EDTA at 10 mM | | | |
| Meropenem with AVI at 4 and | ≤0.002-≤0.002 | ≤0.002 | ≤0.002 |
| EDTA at 20 mM | | | |
| Meropenem with AVI at 8 and | ≤0.002-≤0.002 | ≤0.002 | ≤0.002 |
| EDTA at 10 mM | | | |
| Meropenem with AVI at 8 and | ≤0.002-≤0.002 | ≤0.002 | ≤0.002 |
| EDTA at 20 mM | | | |

### Example 9

Effects of body fluids on the minimum inhibitory concentration (MIC) of Meropenem in combination with Avibactam and EDTA

Objective: The combinations of meropenem (MEM) in the presence of different concentrations of avibactam (AVI) and EDTA were tested. The triple combination test compounds were investigated for in-vitro activity in presence cation-adjusted Mueller Hinton broth (CA-MHB) and CA-MHB supplemented with different body fluids i.e. human serum (25% and 50%), pooled urine (25% and 50%), bovine Surfactant (1 and 5%) and human peritoneal fluid (25 and 50%) against the a set of Gram-negatives clinical isolates from India.

Method: The tolerance screening was performed on a total of 60 isolates to ascertain tolerance to body fluids and total 19 isolates including ATCC strains and non-duplicate clinically relevant Escherichia coli (n=6), Klebsiella pneumoniae (n=3), Pseudomonas aeruginosa (n=6) Acinetobacter baumannii (n=3) and Enterobacter cloacae (n=1) were selected prior to susceptibility testing. Tested compounds included Meropenem with AVI at 4 and EDTA at 10 mM (MEM/A4/E10), Meropenem with AVI at 4 and EDTA at 20 mM (MEM/A4/E20), Meropenem with AVI at 8 and EDTA at 10 mM (MEM/A8/E10), Meropenem with AVI at 8 and EDTA at 20 mM (MEM/A8/E20), Meropenem (MEM), Ceftriaxone with sulbactam at 4 and EDTA at 10 mM (CRO/S10/E10), Ceftazidime-avibactam (CZA), Amikacin (AMK), Tigecycline and Minocycline. The clinical isolates were collected from hospitalized patients from tertiary care medical centers in India. Isolates were tested for susceptibility to different test combinations in the CA-MHB media and CA-MHB media supplemented with different body fluids by broth microdilution in line with Clinical and Laboratory Standards Institute (CLSI) recommendations.

### Results:

Against all tested isolates, the MEM MIC was >32 μg/mL, whereas the combination, MEM/AVI [4/8 mg/L]/EDTA [10 or 20 mM], displayed potent activity in CA-MHB and MIC range was ≤0.002 μg/mL-0.25 μg/mL in CA-MHB media against E. coli producing CTX-M and NDM β-lactamases and K. pneumoniae, respectively (Table 1, 2, 3 and 4).
Similarly, MEM/AVI [4/8 mg/L] and EDTA [10/20 mM] displayed potent activity in CA-MHB media with MIC of ≤0.002 μg/mL against MEM-NS A. baumannii producing OXA/NDM, co-producing NDM and OXA. and P. aeruginosa producing NDM/VIM (Table 1, 2, 3 and 4).
However, with addition of different concentrations of the body fluids in CA-MHB media there was no adverse effect on antibacterial activity was observed with test combinations against E. coli, K. pneumoniae, P. aeruginosa A. baumannii and E. cloacae included in the study.

TABLE 1
In-vitro activity of Meropenem/EDTA 10 mM/Avibactam [4 mg/L] in Body Fluids

| | | CA- | CA- | CA- | CA- | | | CA-MHB + | CA-MHB + |
|---|---|---|---|---|---|---|---|---|---|
| | | MHB + | MHB + | MHB + | MHB + | CA-MHB + | CA-MHB + | Human | Human |
| | | Humann | Humann | Pooled | Pooled | Bovine | Bovine | peritoneal | peritoneal |
| | CA- | serum | serum | urine | urine | Surfactant | Surfactant | fluid | fluid |
| Organism | MHB | 25% | 50% | 25% | 50% | 1% | 5% | 25% | 50% |
| A. baumannii | ≤0.002 | ≤0.002 | ≤0.002 | ≤0.002 | ≤0.002 | ≤0.002 | ≤0.002 | ≤0.004 | ≤0.004 |
| A. baumannii | ≤0.002 | ≤0.002 | ≤0.002 | ≤0.002 | ≤0.002 | ≤0.002 | ≤0.002 | ≤0.004 | ≤0.004 |
| A. baumannii | ≤0.002 | ≤0.002 | ≤0.002 | ≤0.002 | ≤0.002 | ≤0.002 | ≤0.002 | ≤0.004 | ≤0.004 |
| E. coli | ≤0.002 | ≤0.002 | ≤0.002 | ≤0.002 | ≤0.002 | ≤0.002 | ≤0.002 | ≤0.004 | ≤0.004 |
| E. coli | ≤0.002 | ≤0.002 | ≤0.002 | ≤0.002 | ≤0.002 | ≤0.002 | ≤0.002 | | |
| E. coli | 0.12 | ≤0.002 | ≤0.002 | 0.12 | 0.12 | 0.06 | 0.015 | ≤0.004 | ≤0.004 |
| E. coli | ≤0.002 | ≤0.002 | ≤0.002 | ≤0.002 | ≤0.002 | ≤0.002 | ≤0.002 | | |
| E. coli | ≤0.002 | ≤0.002 | ≤0.002 | ≤0.002 | ≤0.002 | ≤0.002 | ≤0.002 | ≤0.004 | ≤0.004 |
| E. coli | ≤0.002 | ≤0.002 | ≤0.002 | ≤0.002 | ≤0.002 | ≤0.002 | ≤0.002 | ≤0.004 | ≤0.004 |
| K. | 0.06 | ≤0.002 | | ≤0.002 | ≤0.002 | ≤0.002 | ≤0.002 | | |
| pneumoniae | | | | | | | | | |
| K. | 0.03 | 0.03 | 0.03 | 0.015 | 0.015 | 0.03 | 0.015 | | |
| pneumoniae | | | | | | | | | |
| K. | 0.25 | ≤0.002 | ≤0.002 | ≤0.002 | 0.06 | 0.03 | 0.06 | ≤0.004 | ≤0.004 |
| pneumoniae | | | | | | | | | |
| K. | 0.015 | 0.008 | 0.008 | 0.03 | 0.03 | 0.015 | 0.015 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.002 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.002 | ≤0.004 | ≤0.004 |
| pneumoniae | | | | | | | | | |
| P. | ≤0.002 | ≤0.002 | ≤0.002 | ≤0.002 | ≤0.002 | ≤0.002 | ≤0.002 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.002 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.002 | ≤0.004 | |
| aeruginosa | | | | | | | | | |
| E. cloacae | 0.015 | ≤0.002 | ≤0.002 | 0.008 | ≤0.002 | 0.008 | ≤0.002 | ≤0.004 | |

No growth in one or more conditions

TABLE 2
In-vitro activity of Meropenem/EDTA 20 mM/Avibactam [4 mg/L] in Body Fluids

| | | CA- | CA- | CA- | CA- | CA- | CA- | CA- | CA- |
|---|---|---|---|---|---|---|---|---|---|
| | | MHB + | MHB + | MHB + | MHB + | MHB + | MHB + | MHB + | MHB + |
| | | Humann | Humann | Pooled | Pooled | Bovine | Bovine | Human | Human |
| | CA- | serum | serum | urine | urine | Surfactant | Surfactant | peritoneal | peritoneal |
| Organism | MHB | 25% | 50% | 25% | 50% | 1% | 5% | fluid 25% | fluid 50% |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | | | |
| E. coli | 0.25 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | ≤0.004 | ≤0.004 | ≤0.004 |
| K. | ≤0.002 | ≤0.002 | | ≤0.002 | ≤0.002 | ≤0.002 | | | |
| pneumoniae | | | | | | | | | |
| K. | 0.06 | ≤0.002 | ≤0.002 | ≤0.002 | 0.03 | 0.03 | | | |
| pneumoniae | | | | | | | | | |
| K. | 0.5 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | 0.008 | ≤0.002 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| P. | ≤0.002 | ≤0.002 | ≤0.002 | ≤0.002 | ≤0.002 | ≤0.002 | ≤0.004 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.004 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.004 | | ≤0.004 |
| aeruginosa | | | | | | | | | |
| E. cloacae | 0.008 | ≤0.002 | ≤0.002 | ≤0.002 | ≤0.002 | ≤0.002 | ≤0.004 | | ≤0.004 |

No growth in one or more conditions

TABLE 3
In-vitro activity of Meropenem/EDTA 10 mM/Avibactam [8 mg/L] in Body Fluids

| | | CA- | CA- | CA- | CA- | CA- | CA- | CA- | CA- |
|---|---|---|---|---|---|---|---|---|---|
| | | MHB + | MHB + | MHB + | MHB + | MHB + | MHB + | MHB + | MHB + |
| | | Humann | Humann | Pooled | Pooled | Bovine | Bovine | Human | Human |
| | CA- | serum | serum | urine | urine | Surfactant | Surfactant | peritoneal | peritoneal |
| Organism | MHB | 25% | 50% | 25% | 50% | 1% | 5% | fluid 25% | fluid 50% |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | ≤0.004 | ≤0.004 | ≤0.004 |
| K. | 0.06 | ≤0.002 | | ≤0.002 | ≤0.002 | ≤0.002 | | | |
| pneumoniae | | | | | | | | | |
| K. | 0.03 | 0.03 | 0.03 | ≤0.002 | 0.03 | 0.015 | | | |
| pneumoniae | | | | | | | | | |
| K. | 0.06 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | 0.004 | 0.015 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| P. | ≤0.002 | ≤0.002 | ≤0.002 | ≤0.002 | ≤0.002 | ≤0.002 | ≤0.004 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.004 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.004 | ≤0.004 | |
| aeruginosa | | | | | | | | | |
| E. cloacae | 0.004 | ≤0.002 | ≤0.002 | ≤0.002 | ≤0.002 | ≤0.002 | ≤0.004 | ≤0.004 | |

No growth in one or more conditions

TABLE 4
Invitro Activity of Meropenem/EDTA 20 mM/Avibactam [8 mg/L] in body fluids

| | | CA- | CA- | CA- | CA- | CA- | CA- | CA- | CA- |
|---|---|---|---|---|---|---|---|---|---|
| | | MHB + | MHB + | MHB + | MHB + | MHB + | MHB + | MHB + | MHB + |
| | | Humann | Humann | Pooled | Pooled | Surfactant | Bovine | Human | Human |
| | CA- | serum | serum | urine | urine | Bovine | Surfactant | peritoneal | peritoneal |
| Organism | MHB | 25% | 50% | 25% | 50% | 1% | 5% | fluid 25% | fluid 50% |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| A. | ≤0.002 | ≤0.002 | ≤0.002 | ≤0.002 | ≤0.002 | ≤0.002 | ≤0.004 | ≤0.004 | ≤0.004 |
| baumannii | | | | | | | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | | | |
| E. coli | ≤0.002 | ≤0.002 | ≤0.002 | ≤0.002 | ≤0.002 | ≤0.002 | ≤0.004 | ≤0.004 | ≤0.004 |
| E. coli | ≤0.002 | ≤0.002 | ≤0.002 | ≤0.002 | ≤0.002 | ≤0.002 | ≤0.004 | ≤0.004 | ≤0.004 |
| K. | ≤0.002 | ≤0.002 | | ≤0.002 | ≤0.002 | ≤0.002 | | | |
| pneumoniae | | | | | | | | | |
| K. | 0.06 | ≤0.002 | ≤0.002 | ≤0.002 | 0.03 | 0.015 | | | |
| pneumoniae | | | | | | | | | |
| K. | 0.12 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| K. | ≤0.002 | ≤0.002 | ≤0.002 | ≤0.002 | ≤0.002 | ≤0.002 | ≤0.004 | ≤0.004 | ≤0.004 |
| pneumoniae | | | | | | | | | |
| P. | ≤0.002 | ≤0.002 | ≤0.002 | ≤0.002 | ≤0.002 | ≤0.002 | ≤0.004 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.004 | ≤0.004 | ≤0.004 |
| aeruginosa | | | | | | | | | |
| P. | ≤0.002 | ≤0.002 | ≤0.002 | ≤0.002 | ≤0.002 | ≤0.002 | ≤0.004 | ≤0.004 | |
| aeruginosa | | | | | | | | | |
| E. cloacae | ≤0.002 | ≤0.002 | ≤0.002 | ≤0.002 | ≤0.002 | ≤0.002 | ≤0.004 | ≤0.004 | |

No growth in one or more conditions

### Conclusion:

5 The triple combination of MEM/AVI [4/8 mg/L]/EDTA [10 or 20 mM] are active in different body fluids and no effect on MICs was observed against E. coli, K. pneumoniae, P. aeruginosa A. baumannii and E. cloacae isolates.

### Example 10

### Minimum Inhibitory Concentration (MIC) Profile of EDTA

Objective: The in-vitro activity of ethylenediaminetetraacetic acid (EDTA) was tested in cation-adjusted Mueller Hinton broth (CA-MHB) and MHB against a panel of Gram-negative clinical isolates from India.

Method: A total of 80 isolates including ATCC strains and non-duplicate clinically relevant Escherichia coli (n=20), Klebsiella pneumoniae (n=20), Pseudomonas aeruginosa (n=21) and Acinetobacter baumannii (n=19) characterized isolates were selected prior to susceptibility testing. The clinical isolates were collected from hospitalized patients from tertiary care medical centers in India. Isolates were tested for susceptibility to different test concentrations of EDTA (1.25, 2.5, 5, 10, 20, 40, 80 mM) in CA-MHB media and MHB by broth microdilution.

### Results:

Against all tested isolates, EDTA MIC₅₀ and MIC₉₀ were 10 mM and >80 mM in CA-MHB, whereas in MHB the MIC₅₀ and MIC₉₀ were 2.5 mM and 80 mM, respectively (Table 1).
EDTA MIC₅₀ and MIC₉₀ against Enterobacterales were 40 mM and >80 mM and against non-fermenters was ≤0.125 mM and ≤0.125 mM in CA-MHB media, respectively.
In MHB media, EDTA MIC₅₀ and MIC₉₀ against Enterobacterales were 10 mM and 80 mM and against non-fermenters was ≤0.125 mM and ≤0.125 mM, respectively.
The MIC₅₀ and MIC₉₀ of EDTA against E. coli were 10 mM and 80 mM, respectively and against K. pneumoniae was 80 mM and >80 mM in CA-MHB, respectively.
EDTA MIC₅₀ and MIC₉₀ against E. coli were 2.5 mM and 20 mM (4-fold lower to CA-MHB) and against K. pneumoniae was 80 mM in MHB (almost similar to CA-MHB).
P. aeruginosa EDTA MIC₅₀ and MIC₉₀ were 10 mM and 20 mM and against A. baumannii a lower MICs of ≤1.25 mM was observed in CA-MHB medium.
A similar EDTA activity was observed for P. aeruginosa and A. baumannii in MHB and CA-MHB media.

TABLE 1
MIC50, MIC90 and MIC ranges for EDTA in CA-MHB
and MHB media against all tested isolates

MIC (mM)

| | | | | |
|---|---|---|---|---|
| Species/Compound | MIC50 | MIC90 | MIN | MAX |

All isolates (n = 80)

| | | | | |
|---|---|---|---|---|
| EDTA CA-MHB | 10 | >80 | ≤1.25 | >80 |
| EDTA MHB | 2.5 | 80 | ≤1.25 | >80 |

Enterobacterales (n = 40)

| | | | | |
|---|---|---|---|---|
| EDTA CA-MHB | 40 | >80 | 2.5 | >80 |
| EDTA MHB | 10 | 80 | ≤1.25 | >80 |

E. coli (n = 20)

| | | | | |
|---|---|---|---|---|
| EDTA CA-MHB | 10 | 80 | 2.5 | >80 |
| EDTA MHB | 2.5 | 20 | ≤1.25 | 80 |

K. pneumoniae (n = 20)

| | | | | |
|---|---|---|---|---|
| EDTA CA-MHB | 80 | >80 | 20 | >80 |
| EDTA MHB | 80 | 80 | 5 | >80 |

Non-fermenters (n = 40)

| | | | | |
|---|---|---|---|---|
| EDTA CA-MHB | ≤1.25 | 20 | ≤1.25 | 20 |
| EDTA MHB | ≤1.25 | 10 | ≤1.25 | 20 |

A. baumannii (n = 19)

| | | | | |
|---|---|---|---|---|
| EDTA CA-MHB | ≤1.25 | ≤1.25 | ≤1.25 | ≤1.25 |
| EDTA MHB | ≤1.25 | ≤1.25 | ≤1.25 | ≤1.25 |

P. aeruginosa (n = 21)

| | | | | |
|---|---|---|---|---|
| EDTA CA-MHB | 10 | 20 | ≤1.25 | 20 |
| EDTA MHB | 10 | 20 | ≤1.25 | 20 |

### Conclusion:

The MIC₉₀ of EDTA against all tested isolates was >80 mM and 80 mM in CA-MHB and MHB medium, respectively. EDTA may or may not have activity against E. coli, K. pneumoniae, P. aeruginosa and A. baumannii irrespective of presence of cations in MHB and type of resistance mechanism.

### Example 11

### Frequency of Resistance (FOR) Study

Objective: The in-vitro study was conducted to determine the frequency of resistance in 8 characterized isolates.

### Method:

Isolates: 8 isolates-E. coli (3), K. pneumoniae (2), A. baumannii (1) and P. aeruginosa (2)
Compound tested: Meropenem; Meropenem Avibactam (4); Meropenem EDTA (10 mM); Meropenem Avibactam (4) EDTA (10 mM)
Antibiotic Concentration used:

| | | Antibiotics concentration [μg/mL] | | | |
|---|---|---|---|---|---|
| | | | Mero | | Mero/ |
| | | | EDTA | Mero/ | Avi [4] |
| IHMA # | # | Meropenem | 10 mM | Avi [4] | EDTA10 mM |
| 1. E. Coli | 4x | 128 | 32 | 32 | 16 |
| | 8x | 256 | 64 | 64 | 32 |
| | 16x | 512 | 128 | 128 | 64 |
| 2 - P. | 4x | 128 | 2 | 32 | 2 |
| aeruginosa | 8x | 256 | 4 | 64 | 4 |
| | 16x | 512 | 8 | 128 | 8 |
| 3 - P. | 4x | 128 | 32 | 32 | 16 |
| aeruginosa | 8x | 256 | 64 | 64 | 32 |
| | 16x | 512 | 128 | 128 | 64 |
| 4 - E. coli | 4x | 128 | 32 | 32 | 16 |
| | 8x | 256 | 64 | 64 | 32 |
| | 16x | 512 | 128 | 128 | 64 |
| 5 - E. coli | 4x | 64 | 16 | 32 | 8 |
| | 8x | 128 | 32 | 64 | 16 |
| | 16x | 256 | 64 | 128 | 32 |
| 6 - K. | 4x | 128 | 128 | 32 | 32 |
| pneumoniae | 8x | 256 | 256 | 64 | 64 |
| | 16x | 512 | 512 | 128 | 128 |
| 7 - K. | 4x | 128 | 64 | 32 | 32 |
| pneumoniae | 8x | 256 | 128 | 64 | 64 |
| | 16x | 512 | 256 | 128 | 128 |
| 8 - A. | 4x | 128 | 0.06 | 32 | 0.008 |
| baumannii | 8x | 256 | 0.12 | 64 | 0.016 |
| | 16x | 512 | 0.25 | 128 | 0.03 |

### Results:

| | | 24 H of Incubation | | | |
|---|---|---|---|---|---|
| | | | Mero- | Mero- | Mero/ |
| | ATB | | penem | penem/ | Avi[4] |
| | concen- | Mero- | EDTA | Avibactam | EDTA |
| Strain | tration | penem | 10 mM | [4] | 10 mM |
| 1 - E. coli | 4x | 0 | 0 | 0 | 0 |
| | 8x | 0 | 0 | 0 | 0 |
| | 16x | 0 | 0 | 0 | 0 |
| 2 - P. | 4x | TMTC | 0 | TMTC | 0 |
| aeruginosa | 8x | TMTC | 0 | TMTC | 0 |
| | 16x | TMTC | 0 | TMTC | 0 |
| 3 - P. | 4x | TMTC | 0 | TMTC | 0 |
| aeruginosa | 8x | TMTC | 0 | TMTC | 0 |
| | 16x | TMTC | 0 | TMTC | 0 |
| 4 - E. coli | 4x | TMTC | 0 | TMTC | 0 |
| | 8x | TMTC | 0 | TMTC | 0 |
| | 16x | TMTC | 0 | TMTC | 0 |
| 5 - E. coli | 4x | 0 | 0 | 0 | 0 |
| | 8x | 0 | 0 | 0 | 0 |
| | 16x | 0 | 0 | 0 | 0 |
| 6 - K. | 4x | TMTC | 0 | TMTC | 0 |
| pneumoniae | 8x | 303 | 0 | TMTC | 0 |
| | 16x | 0 | 0 | 309 | 0 |
| 7 - K. | 4x | TMTC | 0 | TMTC | 0 |
| pneumoniae | 8x | TMTC | 0 | TMTC | 0 |
| | 16x | TMTC | 0 | TMTC | 0 |
| 8 - A. | 4x | TMTC | 0 | TMTC | 0 |
| baumannii | 8x | TMTC | 0 | TMTC | 0 |
| | 16x | TMTC | 0 | TMTC | 0 |

TMTC—To more to count

Conclusion: Tested compound Meropenem Avibactam (4) EDTA (10) has low propensity to develop resistance against the tested isolates.

### Example 12

### Time Kill Assay (TKA) Study

Objective: The in-vitro Time Kill Assay study was conducted to determine the log reduction in 5 characterised MDR isolates.

### Method:

Isolates: 5 isolates-E. coli (3), K. pneumoniae (2)
Compound tested: Meropenem; Meropenem Avibactam (4); Meropenem EDTA (10 mM); Meropenem Avibactam (4) EDTA (10 mM)
1 log—90% killing, 2 log 99% killing and 3 log 99.9% killing. Bactericidal: 3 log reduction (99.9% killing)
MIC:

| | MIC [μg/mL] | | | |
|---|---|---|---|---|
| | | Meropenem/ | | |
| | Meropenem | Avibactam | | Meropenem/ |
| | EDTA | [4] | | Avibactam |
| Organism | 10 mM | EDTA 10 mM | Meropenem | [4] |
| CLSI Range | — | — | 0.008-0.06 | — |
| [μg/mL] | | | | |
| E. coli | ≤0.015 | ≤0.002 | 0.03 | 0.03 |
| E. coli | 0.03 | ≤0.002 | >32 | >8 |
| E. coli | 0.12 | ≤0.002 | 0.25 | 0.008 |
| K. | 1 | 0.06 | >32 | >8 |
| pneumoniae | | | | |
| K. | 0.03 | 0.015 | >32 | >8 |
| pneumoniae | | | | |

### Results:

For E. coli isolates:

For E. coli isolates:

| Log 10 | 0 | 1 | 2 | 4 | 8 | 24 | 0 | 1 | 2 | 4 | 8 | 24 | 0 | 1 | 2 | 4 | 8 | 24 |
|---|---|---|---|---|---|---|---|---|---|---|---|---|---|---|---|---|---|---|
| [symbol] | 5.94 | 6.55 | 7.47 | [symbol] | [symbol] | 10.17 | 5.89 | 6.34 | 7.27 | 8.38 | 8.91 | 9.04 | 5.89 | 6.34 | 7.27 | [symbol] | 8.91 | 9.04 |
| [symbol] | 5.94 | [symbol] | [symbol] | [symbol] | [symbol] | [symbol] | 5.89 | 6.16 | [symbol] | 7.38 | 8.12 | 8.63 | 5.89 | [symbol] | 6.43 | [symbol] | 8.12 | [symbol] |
| [symbol] | 5.94 | [symbol] | [symbol] | 6.76 | 6.34 | [symbol] | 5.89 | 5.87 | 6.17 | 5.29 | [symbol] | 8.16 | [symbol] | [symbol] | 6.17 | [symbol] | 4.54 | 8.16 |
| [symbol] | 5.94 | 6.12 | 6.02 | [symbol] | [symbol] | [symbol] | 5.89 | [symbol] | 5.94 | 4.20 | [symbol] | 6.24 | 5.89 | [symbol] | [symbol] | 4.20 | [symbol] | 6.24 |
| [symbol] | 5.94 | [symbol] | [symbol] | [symbol] | [symbol] | [symbol] | 5.89 | [symbol] | [symbol] | 3.73 | 2.89 | 2.30 | 5.89 | [symbol] | 5.89 | 1.73 | [symbol] | [symbol] |
| [symbol] | 5.94 | 5.73 | 5.16 | [symbol] | [symbol] | [symbol] | 5.89 | 5.37 | [symbol] | [symbol] | 2.81 | 2.30 | 5.89 | 5.37 | 3.37 | 2.83 | 2.81 | [symbol] |
| [symbol] | 5.94 | 6.17 | [symbol] | [symbol] | [symbol] | [symbol] | 5.89 | 5.85 | 6.24 | 6.46 | [symbol] | 2.45 | 5.89 | [symbol] | 6.24 | [symbol] | [symbol] | 2.45 |
| [symbol] | 5.94 | 6.12 | [symbol] | 6.04 | [symbol] | 4.24 | 5.89 | [symbol] | [symbol] | [symbol] | 4.93 | 4.01 | 5.89 | 5.33 | [symbol] | 5.39 | [symbol] | 4.01 |
| [symbol] | 5.94 | 6.13 | [symbol] | 5.85 | 4.40 | 2.30 | 5.89 | 5.93 | 6.19 | 6.08 | 4.20 | 2.30 | 5.89 | 5.93 | [symbol] | [symbol] | 4.20 | 2.30 |
| [symbol] | 5.94 | [symbol] | [symbol] | [symbol] | [symbol] | [symbol] | 5.89 | 5.83 | 6.15 | 4.98 | 3.53 | 2.30 | 5.89 | [symbol] | 6.15 | 4.98 | [symbol] | 2.30 |
| [symbol] | 5.94 | [symbol] | [symbol] | [symbol] | [symbol] | [symbol] | 5.89 | 6.57 | 4.30 | 3.30 | [symbol] | 8.71 | 5.89 | 6.57 | 4.30 | [symbol] | [symbol] | 8.71 |
| [symbol] | 5.94 | [symbol] | [symbol] | [symbol] | [symbol] | [symbol] | 5.89 | [symbol] | [symbol] | [symbol] | 4.95 | 8.75 | 5.89 | 5.70 | 2.30 | [symbol] | 4.99 | 8.75 |
| [symbol] | 5.94 | [symbol] | 4.00 | [symbol] | [symbol] | [symbol] | 5.89 | 5.04 | 2.30 | 2.30 | 2.30 | 2.30 | 5.89 | 5.04 | 2.30 | 2.30 | 2.30 | 2.30 |
| [symbol] | 5.94 | 4.91 | [symbol] | 2.54 | [symbol] | [symbol] | 5.89 | [symbol] | 2.30 | 2.30 | 2.30 | 2.30 | 5.89 | [symbol] | 2.30 | 2.30 | 2.30 | 2.30 |
| [symbol] | 5.94 | [symbol] | [symbol] | [symbol] | [symbol] | [symbol] | 5.89 | [symbol] | [symbol] | 4.30 | 3.20 | 7.40 | 5.89 | 6.09 | 5.84 | 4.30 | 3.20 | 7.40 |
| [symbol] | 5.94 | [symbol] | [symbol] | [symbol] | [symbol] | [symbol] | 5.89 | 6.31 | 4.30 | 3.30 | [symbol] | 6.16 | 5.89 | [symbol] | 4.30 | 3.30 | [symbol] | 6.16 |
| [symbol] | 5.94 | [symbol] | [symbol] | [symbol] | 2.30 | 2.30 | 5.89 | 6.22 | 4.30 | 3.30 | 2.30 | 3.66 | [symbol] | 6.22 | 4.30 | [symbol] | 2.30 | [symbol] |
| [symbol] | 5.94 | 5.31 | [symbol] | [symbol] | [symbol] | [symbol] | 5.89 | [symbol] | 4.30 | 3.30 | 2.30 | 2.30 | 5.89 | [symbol] | 4.30 | 3.30 | 2.30 | 2.30 |
| [symbol] | 4.94 | 4.94 | 4.94 | 4.94 | 4.94 | 4.94 | 4.89 | 4.89 | 4.89 | 4.89 | 4.89 | 4.89 | 4.89 | 4.89 | 4.89 | 4.89 | 4.89 | 4.89 |
| [symbol] | 3.94 | 3.94 | 3.94 | 3.94 | 3.94 | 3.94 | 3.89 | 3.89 | 3.89 | 3.89 | 3.89 | 3.89 | 3.89 | 3.89 | 3.89 | 3.89 | 3.89 | 3.89 |
| [symbol] | 2.94 | 2.94 | 2.94 | 2.94 | 2.94 | 2.94 | 2.89 | 2.89 | 2.89 | 2.89 | 2.89 | 2.89 | 2.89 | 2.89 | 2.89 | 2.89 | 2.89 | 2.89 |

| | | |
|---|---|---|
| Isolate 1 | Isolate 2 | Isolate 3 |
| ATCC 25922 | CTX-M-15; NDM-5; | CTX-M-15; TEM-OSBL; |
| | TEM-OSBL | OXA-181 |

[symbol]

indicates data missing or illegible when filed

For K. pneumoniae isolates:

| Log 10 | 0 | 1 | 2 | 4 | 8 | 24 | 0 | 1 | 2 | 4 | 8 | 24 |
|---|---|---|---|---|---|---|---|---|---|---|---|---|
| [symbol] | 6.59 | 6.90 | 7.77 | 8.76 | 9.04 | 9.27 | 6.27 | 6.82 | 7.68 | 8.70 | 8.99 | 9.02 |
| [symbol] | 6.59 | 6.92 | 7.10 | 8.10 | 8.53 | 8.80 | 6.27 | 6.73 | 7.39 | 8.12 | 8.57 | 8.80 |
| [symbol] | 6.59 | 6.05 | 5.71 | 5.37 | 4.33 | 6.46 | 6.27 | 6.47 | [symbol] | 5.75 | 5.72 | 5.18 |
| [symbol] | 6.59 | 5.70 | 5.18 | 3.30 | 2.30 | 2.30 | 6.27 | [symbol] | 6.13 | 5.10 | 2.30 | 5.19 |
| [symbol] | 6.59 | 5.33 | 4.64 | 2.60 | 2.30 | 4.55 | 6.27 | 6.48 | 5.87 | 4.30 | 2.30 | 2.30 |
| [symbol] | 6.59 | 4.15 | 2.90 | [symbol] | 2.30 | 2.30 | 6.27 | 6.23 | 5.38 | 2.30 | 2.78 | 2.30 |
| [symbol] | 6.59 | 6.61 | 6.42 | 5.29 | 4.82 | 6.10 | 6.27 | [symbol] | 5.80 | 6.20 | 6.52 | 7.37 |
| [symbol] | 6.59 | 6.57 | 6.23 | 4.64 | 3.60 | 3.60 | 6.27 | 6.45 | 6.43 | 5.34 | 4.70 | 2.30 |
| [symbol] | 6.59 | 6.49 | 5.67 | 4.24 | 3.30 | 2.30 | 6.27 | 6.29 | 6.16 | 2.30 | 3.37 | 2.30 |
| [symbol] | 6.59 | 6.40 | 5.15 | 3.39 | 3.30 | 2.30 | 6.27 | 6.07 | 5.51 | 4.60 | 2.50 | 3.22 |
| [symbol] | 6.59 | 5.47 | 3.99 | 2.30 | 5.23 | 8.94 | 6.27 | 4.77 | 4.77 | 4.11 | 6.78 | 8.90 |
| [symbol] | 6.59 | 4.46 | 3.35 | 2.30 | 2.30 | 2.30 | 6.27 | 4.92 | 3.60 | 3.40 | 4.53 | 8.87 |
| [symbol] | 6.59 | 3.30 | 2.60 | 2.30 | 2.30 | 2.30 | 6.27 | 3.30 | 3.30 | 2.30 | 2.30 | 6.58 |
| [symbol] | 6.59 | 2.30 | 2.30 | 2.30 | 2.30 | 2.30 | 6.27 | 2.30 | 2.30 | 2.30 | 2.30 | 2.30 |
| [symbol] | 6.59 | 5.93 | 5.54 | 3.12 | 5.44 | 8.89 | 6.27 | 5.62 | 4.38 | 3.30 | 5.50 | 8.53 |
| [symbol] | 6.59 | 5.84 | 4.25 | 2.30 | 4.51 | 8.94 | 6.27 | 4.81 | 4.61 | 3.30 | 5.14 | 8.57 |
| [symbol] | 6.59 | 5.54 | 3.84 | 2.48 | 3.18 | 8.70 | 6.27 | 4.94 | 4.84 | 4.18 | 5.79 | 8.73 |
| [symbol] | 6.59 | 3.78 | 2.70 | 2.30 | 2.30 | 2.30 | 6.27 | 4.30 | 2.30 | 2.30 | 2.30 | 5.70 |
| −1 log | 5.59 | 5.59 | 5.59 | 5.59 | 5.59 | 5.59 | 5.27 | 5.27 | 5.27 | 5.27 | 5.27 | 5.27 |
| −2 log | 4.59 | 4.59 | 4.59 | 4.59 | 4.59 | 4.59 | 4.27 | 4.27 | 4.27 | 4.27 | 4.27 | 4.27 |
| −3 log | 3.59 | 3.59 | 3.59 | 3.59 | 3.59 | 3.59 | 3.27 | 3.27 | 3.27 | 3.27 | 3.27 | 3.27 |

| | |
|---|---|
| Isolate 1 | Isolate 2 |
| DHA-TRUNC; CTX-M-15; | CTX-M-15; NDM-5; SHV-OSBL; |
| NDM-4; TEM-OSBL; OXA-181 | TEM-OSBL |

[symbol]

indicates data missing or illegible when filed

Conclusion: Tested compound (triple combination of MEM/A VI [4/8 mg/L]/EDTA [10 or 20 mM]) showed a profound log reduction/killing of all the tested isolates in a time and concentration dependent manner for E. coli, Klebsiella isolates.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to fall within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

1.-25. (canceled)

26. A pharmaceutical composition comprising (a) an effective amount of meropenem, (b) an effective amount of avibactam, and (c) an effective amount of EDTA.

27. The pharmaceutical composition as claimed in claim 26, wherein meropenem is present in an amount ranging from 1 gram to 10 gram, avibactam is present in an amount ranging from 1 gram to 5 gram and EDTA is present in an amount ranging from 100 mg to 300 mg.

28. The pharmaceutical composition as claimed in claim 27, wherein the said combination comprises meropenem, avibactam, and EDTA in any one of the following amounts:
(i) about 3 gram of meropenem, about 1.5 gram of avibactam, and about 111 mg of EDTA; or
(ii) about 3 gram of meropenem, about 1.5 gram of avibactam, and about 222 mg EDTA; or
(iii) about 3 gram of meropenem, about 3 gram of avibactam, and about 111 mg EDTA; or
(iv) about 3 gram of meropenem, about 3 gram of avibactam, and about 222 mg EDTA; or
(v) about 6 gram of meropenem, about 1.5 gram of avibactam, and about 111 mg EDTA; or
(vi) about 6 gram of meropenem, about 3 gram of avibactam, and about 222 mg EDTA.

29. The pharmaceutical composition of claim 26 further comprising one or more pharmaceutically acceptable carriers or excipients or the like.

30. A method of treating bacterial infection in a subject, said method comprising administering to said subject an effective amount of (a) meropenem, (b) avibactam, and (c) EDTA.

31. The method as claimed in claim 30, further comprising administering to said subject a daily dose of: (a) 1 gram to 10 gram meropenem, (b) 1 gram to 5 gram avibactam, and (c) 100 mg to 300 mg EDTA.

32. The method as claimed in claim 31, said method comprising administering to said subject a daily dose of: (a) meropenem, (b) avibactam, and (c) EDTA in any one of the following amounts:
(i) about 3 gram of meropenem, about 1.5 gram of avibactam and about 111 mg EDTA; or
(ii) about 3 gram of meropenem, about 1.5 gram of avibactam and about 222 mg EDTA; or
(iii) about 3 gram of meropenem, about 3 gram of avibactam and about 111 mg EDTA; or
(iv) about 3 gram of meropenem, about 3 gram of avibactam and about 222 mg EDTA; or
(v) about 6 gram of meropenem; about 1.5 gram of avibactam and about 111 mg EDTA; or
(vi) about 6 gram of meropenem, about 3 gram of avibactam and about 222 mg EDTA.

33. A method for treating a bacterial infection in pediatric patients, said method comprising administering to said subject a daily dose of (a) meropenem from about 10 mg/kg to about 40 mg/kg, (b) avibactam from about 10 mg/kg to about 12.5 mg/kg, and (c) EDTA from about 1.85 mg/kg to about 3.7 mg/kg.

34. The method as claimed in claim 30, wherein meropenem is administered before, after, or simultaneously with the administration of avibactam and EDTA.

35. The method as claimed in claim 30, wherein the daily dose of (a) meropenem, (b) avibactam, and (c) EDTA is administered at an interval of 8 hours.

36. The method as claimed in claim 30, wherein the dose of (a) meropenem, (b) avibactam, and (c) EDTA is administered as an intravenous infusion from about 30 minutes to about 120 minutes.

37. The pharmaceutical composition of claim 26, comprising 3-6 grams meropenem, 1.5-3 grams avibactam, 110-225 mg EDTA, and one or more pharmaceutically acceptable excipients.

38. The pharmaceutical composition of claim 26, which is in the form of a dry powder suitable for reconstitution with a liquid, or in the form of an injectable liquid.

39. The pharmaceutical composition of claim 26, which is in the form of a unit dosage form and formulated for delivery to a human subject via intravenous or parenteral.

40. A method of treating an infection, the method comprising administering to a human subject afflicted with an infection, or at risk thereof, with the pharmaceutical composition of claim 26, in an amount and for a period of time effective to treat the infection.

41. The method of claim 40, further comprising at least one of the following features:
wherein the infection is a bacterial infection;
wherein the infection is a bacterial infection caused by resistant bacteria;
wherein the infection is a bacterial infection caused by bacteria producing one or more beta-lactamase enzymes;
wherein the infection is a bacterial infection caused by resistant bacteria comprising at least one of Acinetobacter, Escherichia coli, Pseudomonas aeruginosa, Staphylococcus aureus, Enterobacter, Klebsiella, and Citrobacter;
wherein the infection is a urinary tract infection, complicated urinary tract infections, respiratory tract infection, pneumonia, surgical infection, intraabdominal infection, skin and soft tissue infection, or blood stream infections.

42. The method of claim 40, wherein the administration of the pharmaceutical composition results in a daily dose to the human subject of (a) meropenem from about 10 mg/kg to about 40 mg/kg, (b) avibactam from about 10 mg/kg to about 12.5 mg/kg, and (c) EDTA from about 1.85 mg/kg to about 3.7 mg/kg, preferably the pharmaceutical composition is an intravenous infusion.

43. The method as claimed in claim 33, wherein meropenem is administered before, after, or simultaneously with the administration of avibactam and EDTA.

44. The method as claimed in claim 33, wherein the daily dose of (a) meropenem, (b) avibactam, and (c) EDTA is administered at an interval of 8 hours.

45. The method as claimed in claim 33, wherein the dose of (a) meropenem, (b) avibactam, and (c) EDTA is administered as an intravenous infusion from about 30 minutes to about 120 minutes.
